(19) United States
(12) Patent Application Publication
(10) Pub. No.: US 2006/0051407 A1
(43) Pub. Date: Mar. 9, 2006

(54) Method of treating ischemia-reperfusion injury

(76) Inventors: Yoram Richter, Ramat-Hasharon (IL); Elazer R. Edelman, Brookline, MA (US); Gershon Golomb, Efrat (IL); Haim D. Danenberg, Jerusalem (IL)

Correspondence Address: MORGAN & FINNEGAN, L.L.P., 3 World Financial Center, New York, NY, 10281-2101, US

(21) Appl. No.: 11/190,787

(22) Filed: Jul. 26, 2005

Related U.S. Application Data
(63) Continuation-in-part of application No. 10/871,488, filed on Jun. 18, 2004.
Application No. 10/871,488 is a continuation-in-part of application No. 10/607,623, filed on Jun. 27, 2003.

(51) Int. Cl. A61K 31/663; A61K 31/7072; A61K 31/4745; A61K 31/337; A61K 31/60; A61K 9/127; A61K 33/24

(52) U.S. Cl. 424/450; 514/102; 514/291; 424/649; 424/617; 424/724; 514/50; 514/165; 514/169; 514/410; 514/449

(57) ABSTRACT

The present invention relates to methods and compositions designed for the prevention, reduction, treatment or management of ischemia-reperfusion injury. The methods of the invention comprise the administration of an effective amount of a therapeutic formulation containing one or more active compounds in a formulation which specifically decreases or inhibits the activity of and/or eliminates or diminishes the amount of phagocytic cells including, but not limited to, macrophages and/or monocytes. In preferred embodiments, the active compound is a bisphosphonate. The invention also provides pharmaceutical compositions of therapeutic formulations for administration to subjects currently suffering from, having recently suffered, or at risk of suffering from an ischemia-reperfusion injury.

This application is a continuation-in-part of U.S. application Ser. No. 10/871,488 filed Jun. 18, 2004 which is a continuation-in-part of U.S. application Ser. No. 10/607,623 filed Jun. 27, 2003, each of which is incorporated by reference herein in its entirety.

### 1. FIELD OF INVENTION

The present invention relates to methods and compositions designed for the treatment or management of ischemia-reperfusion injury (IRI). The methods of the invention comprise the administration to a patient in need thereof of an effective amount of one or more therapeutic formulations containing an active compound in a formulation which specifically decreases or inhibits the activity of and/or eliminates or diminishes the amount of phagocytic cells including, but not limited to, macrophages and monocytes.

### 2. BACKGROUND OF THE INVENTION

Ischemic injury to vital organs contributes significantly to morbidity and mortality throughout the world. Deprived of oxygen-carrying blood, cellular respiration slows down with damage occurring within minutes. Rapid restoration of circulation, while essential to maintain life, brings its own hazards. Reperfusion produces an inflammatory response that both heightens local damage and leads to systemic insult as well. Acute events such as myocardial infarction, stroke, and cardiac arrest can produce IRI. However, many types of planned surgical procedures, such as organ transplantation and aneurysm repair may require ischemic periods of time during the procedure and therefore also produce IRI events.

The presence of inflammatory cells in the ischemic tissues has traditionally been believed to represent the pathophysiological response to injury. However, experimental studies have shown that while crucial to healing, the influx of inflammatory cells into tissues, specifically macrophages which are phagocytic cells, results in tissue injury beyond that caused by ischemia alone. Such an injury can affect a variety of tissues such as the heart, brain, liver, spleen, intestines, lungs, and pancreas.

Various methods of limiting reperfusion injury have been described such as induced hypothermia, controlled reperfusion, and ischemic preconditioning. Induced hypothermia is the induction of moderate hypothermia (28° C. to 32° C.) in a patient. Mild hypothermia is thought to suppress many of the chemical reactions associated with reperfusion injury. Despite these potential advantages, hypothermia can also produce adverse effects, including arrhythmias, infection, and coagulopathy. Controlled reperfusion refers to controlling the initial period of reperfusion by reperfusing the tissue at a low pressure using blood that has been modified to be hyperosmolar, alkalotic, and substrate-enriched. Ischemic preconditioning is the purposeful causing of short ischemic events to have protective effect by slowing cell metabolism during a longer ischemic event. Although theses treatments may be useful in surgical settings (e.g., before or after planned heart surgery), normally it is not feasible to have the controlled, predetermined conditions required.

Macrophages and the Inflammatory Response

Macrophages and other leukocytes infiltrate the area soon after ischemia ensues. Macrophages secrete several cytokines, which stimulate fibroblast proliferation. However, the activated macrophages also secrete cytokines and other mediators that promote tissue damage. Accordingly, the influx of macrophages into the area increases tissue necrosis and expands the zone of infarct. Thus, although the acute phase of inflammation is a necessary response for the healing process, persistent activation is in fact harmful to the infarct area as well as the area surrounding it, the so-called ‘peri-infarct zone’. The inflammatory response that follows ischemia is critical in determining the severity of the resultant damage caused by the activated macrophages. Plasma levels of macrophage chemoattractant protein-1 (MCP-1) are elevated in patients with ischemia-reperfusion injury and neutralization of this chemokine significantly reduces infarct size.

Thus, there exists a need for a treatment for patients suffering from ischemia-reperfusion injury capable of decreasing or blocking the accumulation of and/or the biological function, including secretion of factors, of phagocytic cells (particularly macrophages and monocytes).

### 3. SUMMARY OF THE INVENTION

The present invention relates to methods and compositions designed for the prevention, reduction, treatment or management of ischemia-reperfusion injury (IRI). The methods of the invention comprise the administration of an effective amount of one or more therapeutic formulations comprising an active compound formulated such that it specifically inhibits the activity of and/or diminishes the amount of phagocytic cells including, but not limited to, macrophages and monocytes. Administration of one or more therapeutic formulations according to the methods of the invention acts as an acute treatment aimed at minimizing the damage (e.g., tissue necrosis) resulting from the patient's IRI.

In preferred embodiments, the therapeutic formulation specifically targets macrophages and/or monocytes. Because macrophages and monocytes are phagocytic cells, in these embodiments, the therapeutic formulations are prepared such that they comprise particles and/or particulates which can enter into a cell primarily or exclusively via phagocytosis. The formulation relates to the form in which the active compound may be provided, i.e., it may be formulated into a particle or particulate form. The therapeutic formulation comprises an active compound in a formulation such that the physiochemical properties, e.g. size or charge, of the formulation can be internalized only or primarily by phagocytosis. The therapeutic formulation may comprise an active compound encapsulated or embedded in a particle or a particulate active compound. Once phagocytosed by the target cell, e.g., macrophages and monocytes, the active compound decreases or inhibits the function of and/or destroys the cell. In preferred embodiments, the active compound in the therapeutic formulation is a bisphosphonate. In more preferred embodiments, the bisphosphonate is clondronate or alendronate.

In one embodiment, the present invention relates to a method of preventing, treating or managing an IRI by administering to an individual in need thereof an effective amount of a therapeutic formulation comprising an active compound that is encapsulated in a particle of a specific dimension. The therapeutic formulation targets phagocytic cells by virtue of its particular properties, such as, for example, using size or charge to allow the therapeutic formulation to be taken-up primarily or exclusively by phagocytosis. Once the therapeutic formulation is taken-up by the phagocytic cell, the encapsulated active compound is released and is able to decrease or inhibit the activity of and/or destroy the phagocytic cell.

In another embodiment, the present invention relates to a method of preventing, treating or managing an IRI by administering to an individual in need thereof an effective amount of a therapeutic formulation comprising an active compound embedded in a particle of a specific dimension. The therapeutic formulation specifically targets phagocytic cells by virtue of their particular properties, such as, for example, using size or charge to allow the therapeutic formulation to be taken-up primarily or exclusively by phagocytosis. Once the therapeutic formulation is taken-up by the phagocytic cell, the embedded active compound is released and is able to decrease or inhibit the activity of and/or destroy the phagocytic cell.

In yet another embodiment, the present invention relates to a method of preventing, treating or managing an IRI by administering to an individual in need thereof an effective amount of a therapeutic formulation comprising a particulate active compound. The active compound is made into particulates of a specific dimension. The therapeutic formulation specifically targets phagocytic cells by virtue of its properties, such as, for example, using size or charge to allow the therapeutic formulation to be taken-up primarily or exclusively by phagocytosis. Once inside the phagocytic cells the particulate active compound is able to decrease or inhibit the activity of and/or destroy the phagocytic cell.

In a further embodiment, the present invention includes a pharmaceutical composition for administration to subjects currently suffering from, having recently suffered, or likely to suffer an IRI comprising a therapeutic formulation with an active compound in the formulation selected from the group consisting of an encapsulated, embedded, and particulate together with a pharmaceutically acceptable vehicle, carrier, stabilizer or diluent for the treatment, management, reduction or prevention of an IRI.

The formulation of present invention is preferably in the size range of 0.03-1.0 microns. However, depending on the type of active compound and/or formulation used, the more preferred ranges include, but are not limited to, 0.05-0.75 microns, 0.07-0.5 microns and 0.1-0.3 microns. In preferred embodiments, the formulation of the present invention is greater than 0.07 microns.

### 4. BRIEF DESCRIPTION OF THE FIGURES

FIG. 1 illustrates the effect of liposomal alendronate treatment on the size of infarct area after transient coronary artery occlusion in rabbits. The size of the infarct zone was calculated as the area of the infarcted zone as a % of the left ventricular area supplied by the occluded artery and thus at risk for subsequent infarction. Data are expressed as mean ±SD, with n=4/group and a p value of p<0.05.

FIGS. 2A-2B illustrate the effect of liposomal alendronate treatment on myocardial morphology after reversible coronary occlusion in rabbits. Control rabbits (A) have distorted myocardial morphology while rabbits treated with liposomal alendronate (B) have a more normal myocardial morphology.

FIGS. 3A-3B illustrate the reduction in macrophage infiltration following treatment with liposomal alendronate after reversible coronary occlusion in rabbits. Control rabbits (A) show increased RAM11+macrophage accumulation in the zone of infarct as compared to rabbits treated with liposomal alendronate (B).

### 5. DETAILED DESCRIPTION OF THE INVENTION

Phagocytic cells, particularly macrophages and monocytes, are involved in the cause and/or pathology of ischemia-reperfusion injury (IRI). Once an ischemia occurs, macrophages/monocytes are recruited to the damaged tissue and secrete cytokines and other mediators that promote tissue damage. This results in tissue injury beyond that caused by ischemia alone which increases tissue necrosis thus expanding the zone of infarct, i.e., permanent tissue damage. Although a complete and chronic incapacitation and/or ablation of phagocytic cells is not desirable, such a decrease or inhibition in phagocytic cell activity and/or presence is desirable in the short term during or after an IRI event to stabilize the patient and/or reduce the damage caused by the IRI event.

IRI was first described in the myocardium for the damages seen by myocardial infarction. However, it is now evident that this condition occurs in a wide variety of organs and tissues, including but not limited to, the brain and other nervous tissue such as the retina and spinal cord, liver, stomach, intestines, kidney, lung, skin, skeletal muscle, and pancreas. Therefore, the present invention can be used to prevent, treat, or manage IRI in various organs before, during, and/or after surgery which requires periods of ischemia. It can be used to prevent, treat, or manage IRI associated with myocardial infarction, stroke, or cardiac arrest. It can be used to prevent, treat, or manage bowel infarction, chronic mesenteric ischemia, acute lower extremity ischemia, ischemic bowel disease, and following complex reconstructions for aortic aneurysms or thoracoabdominal aneurysms.

The present invention relates to methods and compositions designed to decrease or inhibit the activity of and/or eliminate or diminish the amount of phagocytic cells (including, but not limited to, macrophages and monocytes) for an acute, short term period during or following an IRI event for the treatment or management of the IRI. The methods of the invention comprise the administration of an effective amount of one or more therapeutic formulations that comprise an active compound in a formulation which specifically decreases or inhibits the activity of and/or eliminates or diminishes the amount of phagocytic cells (including, but not limited to, macrophages and monocytes) in a patient. Administration of one or more therapeutic formulations is contemplated as an acute, short term treatment aimed at stabilization of the patient and/or minimization of the immediate and long term damage from the IRI.

The therapeutic formulations used in the methods of the invention specifically decrease or inhibit the activity of phagocytic cells and/or eliminate or diminish the amount of phagocytic cells in a patient. Specificity of the therapeutic formulations is due to the ability of the formulations of the active compounds to affect only particular cell types (e.g., phagocytic cells such as macrophages and/or monocytes). In preferred embodiments, specificity of the therapeutic formulation for phagocytic cells is due to the physiochemical properties, e.g. size or charge, of the formulation such that it can only or primarily be internalized by phagocytosis. Once phagocytosed and intracellular, the active compound is released from the formulation and inhibits or decreases the activity of the phagocytic cell and/or destroys the phagocytic cell.

The therapeutic formulations of the present invention, e.g., the encapsulated active compounds, embedded active compounds, or particulate active compounds, suppress the inflammatory response by transiently depleting and/or inactivating cells that are important triggers in the inflammatory response, namely macrophages and/or monocytes. The encapsulated active compound, embedded active compound, and/or particulate active compound are taken-up, by way of phagocytosis, by the macrophages and monocytes. In contrast, non-phagocytic cells are relatively incapable of taking up the formulation due to the large dimension and/or other physiochemical properties of the formulation.

The term “phagocytosis” as used herein refers to a preferred means of entry into a phagocytic cell and is well understood in the art. However, the term should be understood to also encompass other forms of endocytosis which may also accomplish the same effect. In particular, it is understood that receptor-mediated endocytosis and other cellular means for absorbing/internalizing material from outside the cell are also encompassed by the methods and compositions of the present invention.

The invention also provides pharmaceutical compositions comprising one or more therapeutic formulations of the invention for administration to subjects currently suffering from, recently having suffered, or likely to suffer an IRI event.

Any disorder due to ischemia-reperfusion injury (IRI) may be prevented, treated, or managed by the methods of the present invention. An IRI can relate to any tissue including, but not limited to, heart, brain, liver, spleen, intestines, lungs, and pancreas, and can be the result of a planned event, such as the ischemia associated with a surgical procedure, or an unplanned event, such as stroke or myocardial infarction.

In one embodiment, the IRI relates to injury to the heart including, but not limited to, myocardial infarction (MI), acute myocardial infarction (AMI), unstable angina, impending or actual plaque rupture, and peripheral vascular disease.

In another embodiment, the IRI relates to injury to the brain including, but not limited to, transient ischemic attacks (TIA), reversible ischemic neurologic deficit (RIND), and cerebrovascular accidents (CVA, e.g., strokes).

In another embodiment, the IRI relates to injury to the liver including, but not limited to, ischemic hepatitis.

In another embodiment, the IRI relates to injury to the spleen including, but not limited to splenic infarction.

In another embodiment, the IRI relates to injury to the intestines including, but not limited to ischemic bowel disease.

In another embodiment, the IRI relates to injury to the lungs including, but not limited to, pneumonitis and pulmonary embolus.

In another embodiment, the IRI relates to injury to the pancreas including, but not limited to, acute pancreatitis.

In another embodiment, the IRI relates to injury to a limb including, but not limited to, Limb Ischemia.

In a particular embodiment, the IRI injury does not relate to the kidney.

### 5.1 Active Compounds

The active compounds used in the therapeutic formulations and in the methods of the invention specifically decrease or inhibit the activity of macrophages and/or monocytes and/or eliminate or diminish the amount of macrophages and/or monocytes in a patient, by virtue of the physiochemical properties, such as size or charge, of the formulation. The active compound may be an intracellular inhibitor, deactivator, toxin, arresting substance and/or cytostatic/cytotoxic substance that, once inside a phagocytic cell such as a macrophage or monocyte, inhibits, destroys, arrests, modifies and/or alters the phagocytic cell such that it cannot function normally and/or survive.

As used herein, the term “active compounds” refers to molecules which are encapsulated, embedded, or particularized to make up all or part of the therapeutic formulation and provide the inactivating/toxic potency to the therapeutic formulation, e.g., inhibits or decreases macrophage and/or monocyte activity and/or eliminates or decreases the amount of macrophages and/or monocytes. Compounds that can be active compounds include, but are not limited to, inorganic or organic compounds; or a small molecule (less than 500 daltons) or a large molecule, including, but not limited to, inorganic or organic compounds; proteinaceous molecules, including, but not limited to, peptide, polypeptide, protein, post-translationally modified protein, antibodies etc.; or a nucleic acid molecule, including, but not limited to, double-stranded DNA, single-stranded DNA, double-stranded RNA, single-stranded RNA, or triple helix nucleic acid molecules. Active compounds can be natural products derived from any known organism (including, but not limited to, animals, plants, bacteria, fungi, protista, or viruses) or from a library of synthetic molecules. Active compounds can be monomeric as well as polymeric compounds.

In preferred embodiments the active compound is a bisphosphonate or analog thereof. The term “bisphosphonate” as used herein, denotes both geminal and non-geminal bisphosphonates. In a specific embodiment, the bisphosphonate has the following formula (I): 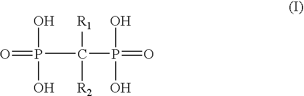
wherein R₁ is H, OH or a halogen atom; and R₂ is halogen; linear or branched C₁-C₁₀ alkyl or C₂-C₁₀ alkenyl optionally substituted by heteroaryl or heterocyclyl C₁-C₁₀ alkylamino or C₃-C₈ cycloalkylamino where the amino may be a primary, secondary or tertiary; —NHY where Y is hydrogen, C₃-C₈ cycloalkyl, aryl or heteroaryl; or R₂ is —SZ where Z is chlorosubstituted phenyl or pyridinyl.

In a specific embodiment, the bisphosphonate is alendronate or an analog thereof. In such an embodiment, the alendronate has the following formula (II):

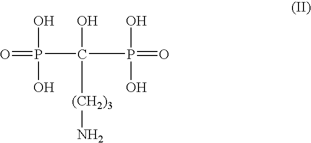

In another specific embodiment, the bisphosphonate is clodronate or an analog thereof. In such an embodiment, the alendronate has the following formula (III):

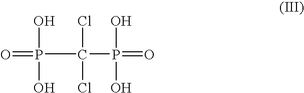

In other specific embodiments, additional bisphosphonates can be used in the methods of the invention. Examples of other bisphosphonates include, but are not limited to, tiludronate, 3-(N,N-dimethylamino)-1-hydroxypropane-1,1-diphosphonic acid, e.g. dimethyl-APD; 1-hydroxy-ethylidene-1,1-bisphosphonic acid, e.g. etidronate; 1-hydroxy-3-(methylpentylamino)-propylidene-bisphosphonic acid, (ibandronic acid), e.g. ibandronate; 6-amino-1-hydroxyhexane-1,1-diphosphonic acid, e.g. amino-hexyl-BP; 3-(N-methyl-N-pentylamino)-1-hydroxypropane-1,1-diphosphonic acid, e.g. methyl-pentyl-APD; 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate; 3-[N-(2-phenylthioethyl)-N-methylamino]-1-hydroxypropane-1,1-bishosphonic acid; 1-hydroxy-3-(pyrrolidin-1-yl)propane-1,1-bisphosphonic acid, 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U81581 (Upjohn); and 1-hydroxy-2-(imidazo[1,2-a]pyridin-3-yl)ethane-1,1-diphosphonic acid, e.g. YM 529, 2-(2-aminopyrimidinio) ethylidene-1,1-bisphosphonic acid betaine (ISA-13-1), or analogs thereof.

The present invention also encompasses therapeutic formulations containing other active compounds that inhibit, destroy, arrest, modify and/or alter the activity or longevity of phagocytic cells including, but not limited to, intracellular inhibitors, intracellular deactivators, intracellular arrestors, intracellular toxins, cytostatic substances, cytotoxic substances, gallium, gold, selenium, gadolinium, silica, mithramycin, sirolimus, paclitaxel, everolimus, and other similar analogs thereof. Generally, chemotherapeutic formulations, such as, for example, 5-fluorouracil, cisplatinum, alkylating agents and other anti-proliferation or anti-inflammatory compounds, such as, for example, steroids, aspirin and non-steroidal anti-inflammatory drugs may also be used as active compounds.

The present invention is meant to encompass the administration of one or more therapeutic formulations in combination to prevent, manage or treat an IRI. The term “in combination” is not limited to the administration of the therapeutic formulations at exactly the same time, but rather it is meant that the therapeutic formulations may be administered to a patient in a sequence and within a time interval such that they can act together to provide an increased benefit than if they were administered otherwise. For example, each therapeutic formulation may be administered at the same time or sequentially in any order at different points in time; however, if not administered at the same time, they should be administered sufficiently close in time so as to provide the desired therapeutic effect. Each therapeutic formulation can be administered separately, in any appropriate form and by any suitable route which effectively transports the therapeutic formulation to the appropriate or desirable site of action.

In various embodiments, the therapeutic formulations are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In one embodiment two or more therapeutic formulations are administered concurrently or within the same patient visit.

The invention provides methods of screening for compounds that can be used as an active compound. Although not intending to be bound by a particular mechanism of action, a compound that is an active compound for use in the methods of the invention can, once targeted to the macrophage and/or monocyte by the physiochemical properties of the formulation itself, i) inhibit phagocyte activity, ii) decrease phagocyte activity, iii) eliminate macrophages/monocytes from circulation, and/or iv) decrease the number of macrophages and/or monocytes in circulation.

The methods of screening for active compounds generally involve incubating a candidate active compounds with phagocytic cells (e.g., macrophages and/or monocytes) either in vitro or in vivo and then assaying for an alteration (e.g., decrease) in phagocytic cell activity or longevity thereby identifying an active compound for use in the present invention. Any method known in the art can be used to assay phagocytic cell activity or longevity.

In one embodiment, phagocytic activity is assayed by the level of cell activation in response to an activating stimulus. For example, macrophage/monocyte activation can be assayed by quantifying the levels of chemotactic factors such as macrophage chemoattractant protein-1 (MCP-1) and macrophage inflammatory protein-1 alpha (MIP-1 alpha) as well as other substances produced by macrophages such as interleukin 1 beta (IL-1β), tissue necrosis factor alpha (TNF-α), histamine, tryptase, PAF, and eicosanoids such as TXA₂, TXB₂, LTB₂, LTB₄, LTC₄, LTD₄, LTE₄, PGD₂ and TXD₄. Any methods known in the art can be used to assay levels of phagocytic secretion products including, but not limited to, ELISA, immunoprecipitation, and quantitative western blot.

In another embodiment, phagocyte longevity is assayed. For example, cell proliferation can be assayed by measuring ³H-thymidine incorporation, by direct cell count, by detecting changes in transcriptional activity of known genes such as proto-oncogenes (e.g., fos, myc) or cell cycle markers; or by trypan blue staining. Any method known in the art can be used to assay for levels of mRNA transcripts (e.g., by northern blots, RT-PCR, Q-PCR, etc.) or protein levels (e.g., ELISA, western blots, etc.).

In one embodiment, a compound that decreases the activity of a phagocytic cell is identified by:

a) contacting a phagocytic cell with a first compound and a second compound, said first compound being a compound which activates said phagocytic cell and said second compound being a candidate compound; and

b) determining the level of activation in said contacted phagocytic cell, wherein a decrease in activation in said contacted cell as compared to the level of activation in a phagocytic cell contacted with said first compound in the absence of said second (i.e., a control cell) indicates that said second compound decreases the activity of a phagocytic cell.

In another embodiment, a compound that decreases the amount of phagocytic cells is identified by:

a) contacting a phagocytic cell with a compound; and

b) determining the viability of said contacted phagocytic cell,

wherein a decrease in viability in said contacted cell as compared to the viability of a phagocytic cell not contacted with said compound (i.e., a control cell) indicates that said compound decreases the amount of phagocytic cells.

In other embodiments, candidate compounds are assayed for their ability to alter phagocytic cell activity or longevity in a manner that is substantially similar to or better than compounds known to alter phagocytic cell activity or longevity in a therapeutically desirable way (e.g., bisphosphonates). As used herein “substantially similar to” refers to a compound having similar action on a phagocytic cell as an exemplified active compound, i.e., a compound that inhibits the activity, function, motility, and/or depletion of phagocytic cells.

Additionally, candidate compounds can be used in animal models of IRIs to assess their ability to be used in the methods of the invention.

### 5.2 Formulation of Active Compounds

Therapeutic formulations comprise active compounds in formulations such that the active compound is in particles that are large enough to only or primarily be internalized by phagocytosis, thus imparting specificity to phagocytic cells such as macrophages and monocytes. Although non-phagocytic cells may be affected by the active compound should it become intracellular, there is no mechanism for a non-phagocytic cell to efficiently internalize the active compound when formulated in this manner (i.e., as a therapeutic formulation). Therapeutic formulations comprise active compounds formulated in the size range of 0.01-1.0 microns, 0.03-1.0 microns, 0.05-0.75 microns, 0.07-0.5 microns, 0.1-0.3 microns, or 0.1-0.18 microns. In one embodiment, the formulation of the present invention is greater than 0.07 microns. However, this is merely an example and other size ranges suitable for phagocytosis by macrophages and/or monocytes may be used without departing from the spirit or scope of the invention.

Any method known in the art can be used to incorporate an active compound into a formulation such that it can only or primarily be internalized via phagocytosis. Formulations of active compounds (i.e., therapeutic formulations) sequester the active compound in an insoluble form for a sufficient time to enhance delivery of the compound to the target site (e.g., the macrophage or monocyte). Furthermore, formulations of active compounds may discharge the compound from the particles when they are within the target site. Thus, only active compounds in an insoluble form (e.g., encapsulated, embedded, or particulate) are present when the therapeutic formulation is extracellular.

The formulation of the active compound is substantially insoluble. Typically, “insoluble” refers to a solubility of one (1) part of a particulate active compound in more than ten-thousand (10,000) parts of a solvent. In one embodiment, the therapeutic formulation is substantially insoluble such that substantially all of the active compound remains in the formulation until after the therapeutic formulation is phagocytosed and is within the phagocytic cell (i.e., intracellular). In another embodiment, the therapeutic formulation is substantially insoluble such that greater than 50%, 60%, 70%, 80%, or 90% of the active compound remains in the formulation after 1 hour, 2 hours, 5 hours, 10 hours, 24 hours, 3 days, 10 days, 30 days, 60 days in a physiologic media (e.g., water, saline, blood, plasma, etc.). In another embodiment, the therapeutic formulation is substantially insoluble such that the active compound is not in a soluble form within the body in levels to substantially effect non-phagocytic cell types.

In one embodiment, the active compound is encapsulated in a particle (i.e., encapsulating agent) of desired properties. In a specific embodiment, the encapsulating agent is a liposome. The liposomes may be prepared by any of the methods known in the art (see, e.g., Mönkkönen, J. et al., 1994, J. Drug Target, 2:299-308; Mönkkönen, J. et al., 1993, Calcif. Tissue Int., 53:139-145; Lasic DD., Liposomes Technology Inc., Elsevier, 1993, 63-105.(chapter 3); Winterhalter M, Lasic DD, Chem Phys Lipids, 1993; 64(1-3):35-43).

Generally, liposomes are formed when thin lipid films or lipid cakes are hydrated and stacks of liquid crystalline bilayers become fluid and swell. The hydrated lipid sheets detach during agitation and self-close to form large, multilamellar vesicles (LMV). Once these particles have formed, reducing the size of the particle requires energy input in the form of sonic energy (sonication) or mechanical energy (extrusion).

Disruption of LMV suspensions using sonic energy (sonication) typically produces small, unilamellar vesicles (SUV). The most common instrumentation for preparation of sonicated particles are bath and probe tip sonicators. Alternatively, lipid extrusion is a technique in which a lipid suspension is forced through a polycarbonate filter with a defined pore size to yield particles having a diameter near the pore size of the filter used.

The liposomes may be positively charged, neutral or, more preferably, negatively charged. The liposomes may be a single lipid layer or may be multilamellar. Suitable liposomes in accordance with the invention are preferably non-toxic liposomes such as, for example, those prepared from phosphatidyl-choline phosphoglycerol and cholesterol. The components of the liposome and/or the amount of each component can be varied using methods known in the art and the formulation which has desirable characteristics (e.g., retention of encapsulated active compound until it is phagocytosed) can be empirically determined.

In a specific embodiment, liposomes are prepared by dissolving distearoylphosphatidylglycerol (DSPG), distearoyl-phosphatidylcholine (DSPC) and cholesterol (in a 1:2:1 ratio) in chloroform: methanol (9:1). After evaporating the solvent, hydrated diisopropylether is added to the solution. The active compound is added before sonication at 55° C. for a period of 45 minutes. The organic phase is then evaporated.

In another embodiment, the active compound is embedded in a particle (i.e., embedding agent) of desired properties. An active compound which is embedded includes those active compounds that are embedded, enclosed, and/or adsorbed within a particle, dispersed in the particle matrix, adsorbed or linked on the particle surface, or a combination of any of these forms. In specific embodiments, the embedding agent is a microparticle, nanoparticle, nanosphere, microsphere, microcapsule, or nanocapsule (see e.g., M. Donbrow in: Microencapsulation and Nanoparticles in Medicine and Pharmacy, CRC Press, Boca Raton, Fla., 347, 1991). Embedding agents include both polymeric and non-polymeric preparations. In a specific embodiment, the embedding agent is a nanoparticle. Nanoparticles can be spherical, non-spherical, or polymeric particles. The active compound may be embedded in the nanoparticle, dispersed uniformly or non-uniformly in the polymer matrix, adsorbed on or linked to the surface, or in combination of any of these forms. In a preferred embodiment, the polymer used for fabricating nanoparticles is biocompatible and biodegradable, such as poly(DL-lactide-co-glycolide) polymer (PLGA). However, additional polymers which may be used for fabricating the nanoparticles include, but are not limited to, PLA (polylactic acid), and their copolymers, polyanhydrides, polyalkyl-cyanoacrylates (such as polyisobutylcyanoacrylate), polyethyleneglycols, polyethyleneoxides and their derivatives, chitosan, albumin, gelatin and the like.

In a specific embodiment, nanoparticles are prepared by a solvent evaporation polymer precipitation technique using a double emulsion system. The active compound and NaHCO₃ are dissolved in Tris buffer. Poly(DL-lactide-co-glycolide) polymer (PLGA) is dissolved in dichloromethane. The aqueous active compound solution is added to the PLGA organic solution and a water in oil (W/O) emulsion is formed by sonication over an ice-bath using a probe type sonicator. This W/O emulsion is then added to a polyvinyl alcohol (PVA) filter sterilized solution, and the pH is adjusted to 7.4 with NaOH solution containing CaCl₂ in a molar ratio of 2:1 to the active compound. The mixture is mixed over an ice bath, forming a double emulsion (W/O/W). The emulsion is stirred at 4° C. overnight to allow evaporation of the organic solvent.

In another embodiment, the active compound is in particulate form, the particles each being of desired properties. A particulate active compound includes any insoluble suspended or dispersed particulate form of the active compound which is not encapsulated, entrapped or absorbed within or on a particle. An active compound which is in particulate form includes those active compounds that are suspended or dispersed insoluble colloids, insoluble aggregates, insoluble flocculates, insoluble salts, insoluble complexes, and insoluble polymeric chains of an active compound. Such particulates are insoluble in the fluid in which they are stored/administered (e.g., saline or water) as well as the fluid in which they provide their therapeutic effect (e.g., blood or serum). Any method known in the art to make particulates or aggregates can be used. Particulates can be any shape.

### 5.3 Determination of Particle Size

Therapeutic formulations comprise active compounds that are formulated such that the size of the particle (e.g., encapsulated, embedded or particularized active compound) is large enough to only or primarily be internalized by phagocytosis, that is, preferably larger than 0.03 microns and more preferably larger than 0.07 microns. In preferred embodiments, such formulations are 0.03-1.0 microns, 0.05-0.75 microns, 0.07-0.5 microns, or 0.1-0.3 microns. Any method known in the art can be used to determine the size of the particles in the therapeutic formulation before administration to a patient in need thereof. For example, a Nicomp Submicron Particle Sizer (model 370, Nicomp, Santa Barbara, Calif.) or a Malvern Zetasizer Nano ZS (model ZS-ZEN3600, Malvern, Worcestershire, United Kingdom) utilizing laser light scattering can be used.

Methods can be used to encapsulate, embed, or particularize active compounds that produce particles of varying sizes, including those smaller or larger than in the preferred embodiments. Any method known in the art may be used to separate the encapsulated, embedded or particularized active compounds that are of the desired size from those that are outside the range (e.g., too small or too large) of the desired size. Therapeutic formulations may include only or primarily those particles of active compound that have been determined to be within a desired size range.

### 5.4 Administration of the Formulation

Effective amounts of the therapeutic formulations of the invention are contemplated as short term, acute therapy and are not meant for chronic administration. Time period of treatment is preferably such that it produces inhibition/depletion of the target phagocytic cells for a period that is less than a month, preferably less than two weeks, most preferably up to one week. Empirically, one can determine this by administering the therapeutic formulation to an individual in need thereof (or an animal model of such an individual) and monitoring the level of inhibition/depletion at different time points. One may also correlate the time of inhibition with the appropriate desired clinical effect, e.g. reduction in the acute risk of IRI.

### 5.5 Characterization of Therapeutic Utility

The term “effective amount” denotes an amount of a therapeutic formulation which is effective in achieving the desired therapeutic result, namely inhibited or decreased phagocytic cell activity and/or elimination or reduction in the amount of phagocytic cells. In one embodiment, the desired therapeutic result of inhibiting or decreasing phagocytic cell activity and/or eliminating or reducing in the amount of phagocytic cells minimizes the infarct size and/or the amount of tissue necrosis in a patient having suffered an IRI.

Toxicity and efficacy of the therapeutic methods of the instant invention can be determined by standard pharmaceutical procedures in cell cultures or experimental animals, e.g., for determining the LD₅₀ (the dose lethal to 50% of the population), the No Observable Adverse Effect Level (NOAEL) and the ED₅₀ (the dose therapeutically effective in 50% of the population). The dose ratio between toxic and therapeutic effects is the therapeutic index and it can be expressed as the ratio LD₅₀/ED₅₀ or NOAEL/ED₅₀. Therapeutic formulations that exhibit large therapeutic indices are preferred. While therapeutic formulations that exhibit toxic side effects may be used, care should be taken to design a delivery system that targets such therapeutic formulations to the site of affected tissue in order to minimize potential damage to unaffected cells and, thereby, reduce side effects.

The data obtained from the cell culture assays and animal studies can be used in determining a range of dosage of the formulation for use in humans. The dosage of such therapeutic formulations lies preferably within a range of circulating concentrations that include the ED₅₀ with little or no toxicity. The dosage may vary within this range depending upon the dosage form employed and the route of administration utilized. For any therapeutic formulation used in the method of the invention, the effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The protocols and compositions of the invention are preferably tested in vitro, and then in vivo, for the desired therapeutic activity, prior to use in humans. One example, of such an in vitro assay is an in vitro cell culture assay in phagocytic cells which are grown in culture, and exposed to or otherwise administered one or more therapeutic formulations, and observed for an effect, e.g., inhibited or decreased activity and/or complete or partial cell death. The phagocytic cells may be obtained from an established cell line or recently isolated from an individual as a primary cell line. Many assays standard in the art can be used to measure the activity of the formulation on the phagocytic cells; for example, macrophage/monocyte activation can be assayed by quantitating the levels of chemotactic factors such as macrophage chemoattractant protein-1 (MCP-1), interleukin 1 beta (IL-1β), tissue necrosis factor alpha (TNF-α) and macrophage inflammatory protein-1 alpha (MIP-1 alpha). Many assays standard in the art can be used to assess survival and/or growth of the phagocytic cells; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining.

Selection of the preferred effective dose can be determined (e.g., via clinical trials) by a skilled artisan based upon the consideration of several factors known to one of ordinary skill in the art. Such factors include the disorder to be prevented, managed or treated, the symptoms involved, the patient's body mass, the patient's immune status and other factors known to the skilled artisan to reflect the accuracy of administered pharmaceutical compositions.

### 5.6 Pharmaceutical Compositions and Routes of Administration

Therapeutic formulations for use in the methods of the invention may be in numerous forms, depending on the various factors specific for each patient (e.g., the severity and type of disorder, age, body weight, response, and the past medical history of the patient), the number and type of active compounds in the formulation, the type of formulation (e.g., encapsulated, embedded, particulate, etc.), the form of the composition (e.g., in liquid, semi-liquid or solid form), and/or the route of administration (e.g., oral, intravenous, intramuscular, intra-arterial, intramedullary, intrathecal, intraventricular, transdermal, subcutaneous, intraperitoneal, intranasal, enteral, parenteral, topical, sublingual, vaginal, or rectal means). Pharmaceutical carriers, vehicles, excipients, or diluents may be included in the compositions of the invention including, but not limited to, water, saline solutions, buffered saline solutions, oils (e.g., petroleum, animal, vegetable or synthetic oils), starch, glucose, lactose, sucrose, gelatin, malt, rice, flour, chalk, silica gel, sodium stearate, glycerol monostearate, talc, sodium chloride, dried skim milk, glycerol, propylene, glycol, ethanol, dextrose and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms.

Pharmaceutical compositions can be administered systemically or locally, e.g., near the site of pathology of an IRI. Additionally, systemic administration is meant to encompass administration that can target to a particular area or tissue type of interest.

Preferred modes of administration include intravenous (IV) and intra-arterial (IA). Other suitable modes of administration include intramuscular (IM), subcutaneous (SC), and intraperitonal (IP) and oral (PO). Such administration may be bolus injections or infusions. Another mode of administration may be by perivascular delivery. The therapeutic formulation may be administered directly or after dilution. Combinations of any of the above routes of administration may also be used in accordance with the invention.

In one embodiment, a pharmaceutical composition containing one or more therapeutic formulations is administered immediately at the onset of the first symptoms of IRI.

In another embodiment, a pharmaceutical composition containing one or more therapeutic formulations is administered immediately after any ischemic event. In a specific embodiment, a pharmaceutical composition containing one or more therapeutic formulations is administered after an ischemic event and prior to reperfusion. In another specific embodiment, a pharmaceutical composition containing one or more therapeutic formulations is administered after an ischemic event and during reperfusion.

In another embodiment, a pharmaceutical composition containing one or more therapeutic formulations may be administered just after onset of symptoms of IRI, for example, within minutes of symptom onset. Alternatively and/or additionally, the compositions may be administered within 1 hour, or about 2 hours, or about 3 hours or about 4 hours, or about 5 hours or about 6 hours, up to within 1-3 days after onset of symptoms.

In another embodiment, a pharmaceutical composition containing one or more therapeutic formulations is administered to a patient with an increased risk of IRI prior to any symptoms of IRI. For example, one or more therapeutic formulations of the invention may be administered to a patient prior to a procedure which increases the risk of IRI such as, for example, an angioplasty procedure (e.g., a percutaneous transluminal coronary angioplasty) which increases the risk of plaque rupture and thus an acute myocardial infarction or myocardial infarction. It may be preferred to administer the composition up to 3 days before such a procedure. Also preferred, administration may be 1-6 hours before the procedure or within 1 hour of the procedure or less than 1 hour before or even within minutes of the procedure. The skilled person can readily determine the appropriate timing of administration depending on various physiological factors, specific to the individual patient, such as, for example, weight, medical history and genetic predisposition, as well as various factors which influence the anticipated risk of plaque rupture such as complexity of the procedure to be performed.

The contents of all published articles, books, reference manuals and abstracts cited herein, are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Modifications and variations of the present invention are possible in light of the above teachings.

### 6. EXAMPLES

The following examples as set forth herein are meant to illustrate and exemplify the various aspects of carrying out the present invention and are not intended to limit the invention in any way.

### 6.1 Effect of Liposomal Bisphosphonate on the Size of the Zone of Infarct

The effects of treatment with encapsulated bisphosphonates on the zone of infarct were studied in a rabbit AMI model. The zone of infarct represents the tissue damage resulting from the IRI event. Liposomal Alendronate, approx. 0.150 μm in diameter was made using the following outline:
a. Dissolve lipids, DSPC, DSPG and cholesterol in 1/1 ethanol/tert-butanol.
b. Dilute solvent into buffer containing Alendronate to generate large multilamellar vesicles (MLVs).
c. Extrude MLVs through 200 nm polycarbonate filters to generate large unilamellar 150±20 nm vesicles (LUVs).
d. Ultra-filtrate LUVs to remove un-encapsulated alendronate.
e. Sterile filter

Eight New Zealand White male rabbits, 2.5-3.5 kg B.W., were fed normal chow and water ad libitum. The rabbits were randomly administered saline (control) or liposomal alendronate (3 mg/kg, i.v.) as a single infusion simultaneous with coronary artery occlusion. The rabbits were anesthetized by Ketamine/Xylazine (35 mg/kg; 5 mg/kg) and Isoflurane. The experiment was performed with respiratory support given by intubation and mechanical ventilation with isoflurane in balance oxygen, and continuous echocardiogram (ECG) and arterial blood pressure (catheter in ear artery) monitoring. Thoracotomy was performed through the left 4^{th} intercostal space, followed by pericardiotomy and creation of a pericardial cradle. The left main coronary artery was identified and a large branch was encircled by a 5-0 silk suture and a snare. Thereafter, the snare was tightened for 30 minutes. Ischemia was verified by ECG changes (ST-T segment elevation), changes of segment coloration and hypokinesia. After thirty minutes, the snare was released and resumption of blood flow was confirmed. The suture was left in place, released, and the chest cavity was closed in layers. Buprenex was administered to the rabbits for analgesia for 2-3 additional days. Following euthanasia with Penthotal, the rabbits were sacrificed after 7 days and the hearts were harvested. The coronary arteries were perfused through the ascending aorta with saline, followed by tightening of the suture on the previously occluded coronary artery and perfusion of the coronary arteries with 0.5% Evans blue solution (Sigma) to stain areas of re-endothelialization (presence of blood). The left ventricular area unstained by Evans blue was defined as the area at risk. The hearts were then frozen at −20° C. for 24 hours and cut into transverse sections 2 mm apart. Slices of the hearts were incubated for 30 minutes in the vital stain tritetrazolium chloride (TTC, 1%, Sigma), fixed in 10% natural buffered formalin to stain cells that had been alive previous to tissue processing. The left ventricular area not stained by TTC (white) was defined as the area of infarct. The stained sections were then photographed and processed by digital planimetry (Photoshop).

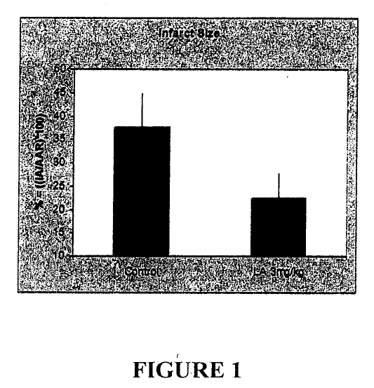

Rabbits treated with liposomal alendronate had a zone of infarct that was 29.5±6% of the area at risk. This was contrasted with the control rabbits (untreated with liposomal alendronate) which showed an infarct zone that was 42±5.5% of the area at risk (FIG. 1). Accordingly, liposomal alendronate was effective in reducing the zone of infarct, thereby reducing tissue damage associated with this IRI event. No adverse effects were observed in the treatment group.

### 6.2 Effect of Liposomal Bisphosphonate On Myocardial Morphology

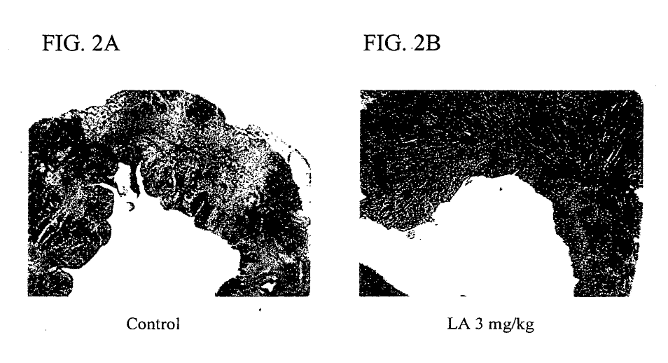

Rabbits as treated in Section 6.1 showed variation in myocardial morphology as exhibited by Hemotoxylin and Eosin staining. The control rabbits have a distorted myocardial morphology (FIG. 2A) while the rabbits treated with liposomal alendronate exhibit a more normal morphology (FIG. 2B).

### 6.3 Effect of Liposomal Bisphosphonate on Macrophage Infiltration

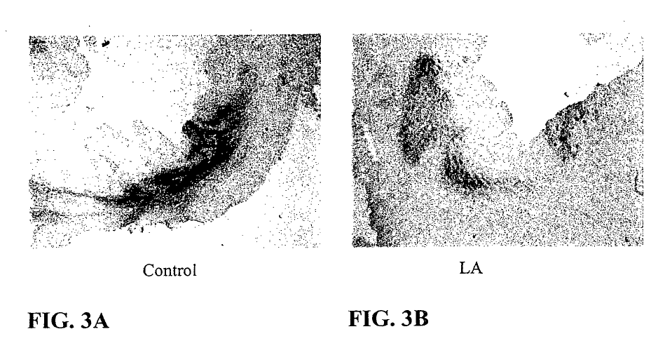

Rabbits as treated in Section 6.1 showed a reduction in macrophage infiltration in rabbits treated with liposomal alendronate. Representative sections of the rabbits' hearts were subjected to immunostaining for RAM11+ macrophages. Sections from rabbits treated with liposomal alendronate (FIG. 3B) showed less staining and therefore had less RAM11+ macrophages accumulation than sections from control rabbits (FIG. 3A).

Liposomal alendronate was also shown to reduce the number of circulating monocytes systemically. Rabbits were administered saline (control) or liposomal alendronate (3 mg/kg, i.v.) Monocyte levels in circulating blood were determined using FACS analysis for CD-14. At 48 hours after injection with liposomal alendronate, the blood monocyte population was reduced by 75-95% as compared to the control group.

What is claimed:

1. A method of treating an ischemia-reperfusion injury comprising administering to a patient in need thereof an effective amount of a formulation comprising an encapsulated active compound, wherein the formulation reduces a zone of infarct, thereby minimizing the damage of the ischemia-reperfusion injury.

2. A method of treating an ischemia-reperfusion injury comprising administering to a patient in need thereof an effective amount of a formulation comprising an embedded active compound, wherein the formulation reduces a zone of infarct, thereby minimizing the damage of the ischemia-reperfusion injury.

3. A method of treating an ischemia-reperfusion injury comprising administering to a patient in need thereof an effective amount of a formulation comprising a particulate active compound, wherein the formulation reduces a zone of infarct, thereby minimizing the damage of the ischemia-reperfusion injury.

4. The method as in one of claims 1-3, wherein the formulation inhibits blood monocyte or tissue macrophage activity.

5. The method as in one of claims 1-3, wherein the formulation decreases blood monocyte or tissue macrophage numbers.

6. The method as in one of claims 1-3, wherein the formulation has a size range of 0.01-1.0 microns.

7. The method as in one of claims 1-3, wherein the formulation has a size range of 0.07-0.5 microns.

8. The method as in one of claims 1-3, wherein the formulation has a size range of 0.1-0.3 microns.

9. The method as in one of claims 1-3, wherein the formulation has a size range of 0.1-0.18 microns.

10. The method as in one of claims 1-3, wherein the active compound is an intra-cellular inhibitor.

11. The method as in one of claims 1-3, wherein the active compound is an intra-cellular deactivator.

12. The method as in one of claims 1-3, wherein the active compound is an intra-cellular arrestor.

13. The method as in one of claims 1-3, wherein the active compound is an intra-cellular toxin.

14. The method as in one of claims 1-3, wherein the active compound is a cytostatic substance.

15. The method as in one of claims 1-3, wherein the active compound is a cytotoxic substance.

16. The method as in one of claims 1-3, wherein the active compound is selected from the group consisting of gallium, gold, selenium, gadolinium, silica, mithramycin, sirolimus, paclitaxel, everolimus, 5-fluorouracil, cisplatinum, steroids, and aspirin.

17. The method as in one of claims 1-3, wherein the active compound is a bisphosphonate.

18. The method of claim 17, wherein said bisphosphonate has formula (I): 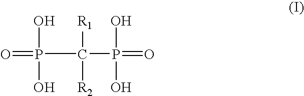
wherein R₁ is H, OH or halogen group; and
R₂ is halogen; linear or branched C₁-C₁₀ alkyl or C₂-C₁₀ alkenyl, optionally substituted by heteroaryl or heterocyclyl C₁-C₁₀ alkylamino or C₃-C₈ cycloalkylamino, where the amino may be a primary, secondary or tertiary amine; —NHY where Y is hydrogen, C₃-C₈ cycloalkyl, aryl or heteroaryl; or —SZ, where Z is chlorosubstituted phenyl or pyridinyl.

19. The method according to claim 17, wherein the bisphosphonate is selected from the group consisting of clodronate, etidronate, tiludronate, pamidronate, alendronate, risendronate, and ISA 13-1.

20. The method of claim 1, wherein the active compound is encapsulated in a liposome.

21. The method of claim 2, wherein the active compound is embedded in a carrier selected from the group consisting of microparticles, nanoparticles, microspheres, and nanospheres.

22. The method of claim 3, wherein the active compound is a particulate selected from the group consisting of aggregates, flocculates, colloids, polymer chains, insoluble salts and insoluble complexes.

23. The method as in one of claims 1-3, wherein the ischemia-reperfusion injury is selected from the group consisting of myocardial infarction, acute myocardial infarction, unstable angina, impending or actual plaque rupture, peripheral vascular disease, transient ischemic attacks, reversible ischemic neurologic deficit, cerebrovascular accidents, ischemic hepatitis, splenic infarction, ischemic bowel disease, limb ischemia, pneumonitis, pulmonary embolus, and acute pancreatitis.

24. A method of treating an ischemia-reperfusion injury followed by tissue necrosis comprising administering to a patient in need thereof an effective amount of a formulation comprising an encapsulated bisphosphonate, thereby minimizing damage resulting from the tissue necrosis.

25. A method of treating an ischemia-reperfusion injury followed by tissue necrosis comprising administering to a patient in need thereof an effective amount of a formulation comprising an embedded bisphosphonate, thereby minimizing damage resulting from the tissue necrosis.

26. A method of treating an ischemia-reperfusion injury followed by tissue necrosis comprising administering to a patient in need thereof an effective amount of a formulation comprising a particulate bisphosphonate, thereby minimizing damage resulting from the tissue necrosis.

27. The method as in one of claims 24-26, wherein the formulation inhibits blood monocyte or tissue macrophage activity.

28. The method as in one of claims 24-26, wherein the formulation decreases blood monocyte or tissue macrophage numbers.

29. The method according to claim 24, wherein the bisphosphonate is encapsulated in a liposome.

30. The method according to claim 25, wherein the bisphosphonate is embedded in a carrier selected from the group consisting of microparticles, nanoparticles, microspheres, and nanospheres.

31. The method according to claim 26, wherein the bisphosphonate particulate is selected from the group consisting of aggregates, flocculates, colloids, polymer chains, insoluble salts and insoluble complexes.

32. The method as in one of claims 1-3 and 24-26, wherein the formulation is administered following an ischemia-reperfusion injury.

33. The method as in one of claims 1-3 and 24-26, wherein the formulation is administered during an ischemia-reperfusion injury.

34. The method as in one of claims 1-3 and 24-26, wherein the formulation is administered prior to the anticipated onset of an ischemia-reperfusion injury.

35. The method as in one of claims 1-3 and 24-26, wherein the formulation is administered during reperfusion.

36. The method as in one of claims 1-3 and 24-26, wherein the formulation is administered prior to or during a procedure where an ischemia-reperfusion injury is probable.

37. The method of claim 36, wherein the procedure is a percutaneous transluminal coronary angioplasty.

38. A method of reducing the zone of infarct following an ischemia-reperfusion injury comprising administering to an individual in need thereof an effective amount of a formulation comprising an encapsulated bisphosphonate.

39. A method of reducing the zone of infarct following an ischemia-reperfusion injury comprising administering to an individual in need thereof an effective amount of a formulation comprising an embedded bisphosphonate.

40. A method of reducing the zone of infarct following an ischemia-reperfusion injury comprising administering to an individual in need thereof an effective amount of a formulation comprising a particulate bisphosphonate.

41. The method according to claim 38, wherein the bisphosphonate is encapsulated in a liposome.

42. The method according to claim 39, wherein the bisphosphonate is embedded in a carrier selected from the group consisting of microparticles, nanoparticles, microspheres, and nanospheres.

43. The method according to claim 40, wherein the bisphosphonate particulate is selected from the group consisting of aggregates, flocculates, colloids, polymer chains, insoluble salts and insoluble complexes.
